(19) United States
(12) Patent Application Publication
(10) Pub. No.: US 2024/0051921 A1
(43) Pub. Date: Feb. 15, 2024

(54) CRYSTALLINE FORM OF A PHENOLIC TRPV1 AGONIST PRODRUG

(71) Applicant: Concentric Analgesics, Inc., San Francisco, CA (US)

(72) Inventor: Susan WOLLOWITZ, San Francisco, CA (US)

(21) Appl. No.: 18/257,272

(22) PCT Filed: Dec. 13, 2021
(86) PCT No.: PCT/US2021/063115

§ 371 Date: Jun. 13, 2023

Related U.S. Application Data
(60) Provisional application No. 63/125,313, filed on Dec. 14, 2020.

(51) Int. Cl. C07D 211/26

(52) U.S. Cl. CPC C07D 211/26; C07B 2200/13

(57) ABSTRACT

Described herein is a crystalline form of (E)-2-methoxy-4-((8-methylnon-6-enamido)methyl)phenyl 2-((methylamino)methyppiperidine-1-carboxylate hydrochloride, and solvates thereof.

### CROSS-REFERENCE

This application claims benefit of U.S. Provisional Patent Application No. 63/125,313, filed on Dec. 14, 2020 which is incorporated herein by reference in its entirety.

### BACKGROUND

Capsaicin (8-methyl-N-vanillyl-6-nonenamide) is a highly selective agonist for transient receptor potential vanilloid 1 receptor (TRPV1; formerly known as vanilloid receptor 1 (VR1)), a ligand-gated, non-selective cation channel. TRPV1 agonists, such as capsaicin, have been shown to diminish pain in various settings, but there are problems associated with their use.

### SUMMARY OF THE INVENTION

Described herein is (E)-2-methoxy-4-((8-methylnon-6-enamido)methyl)phenyl 2-((methylamino)methyl)piperidine-1-carboxylate, including pharmaceutically acceptable salts, solvates (including hydrates), polymorphs, and amorphous phases, and methods of uses thereof. In some embodiments, (E)-2-methoxy-4-((8-methylnon-6-enamido)methyl)phenyl 2-((methylamino)methyl)piperidine-1-carboxylate is used in the manufacture of medicaments for the treatment of pain.

Also described herein are methods for preparing crystalline forms of (E)-2-methoxy-4-((8-methylnon-6-enamido)methyl)phenyl 2-((methylamino)methyl)piperidine-1-carboxylate. Further described are pharmaceutical compositions that include the crystalline forms of (E)-2-methoxy-4-((8-methylnon-6-enamido)methyl)phenyl 2-((methylamino)methyl)piperidine-1-carboxylate and methods of using the compound in the treatment of diseases or conditions.

In one aspect, described herein is a crystalline form of (E)-2-methoxy-4-((8-methylnon-6-enamido)methyl)phenyl 2-((methylamino)methyl)piperidine-1-carboxylate hydrochloride, or a solvate thereof.

In some embodiments, the crystalline form of (E)-2-methoxy-4-((8-methylnon-6-enamido)methyl)phenyl 2-((methylamino)methyl)piperidine-1-carboxylate hydrochloride is Form 1 having at least one of the following properties:
(a) an X-ray powder diffraction (XRPD) pattern substantially the same as shown in FIG. 1;
(b) an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with a broad endotherm having an onset at about 71.5° C.;
(f) a water content from about 0-20% wt; or
(g) combinations thereof.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has an X-ray powder diffraction (XRPD) pattern substantially the same as shown in FIG. 1.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has a thermo-gravimetric analysis (TGA) substantially similar to the one set forth in FIG. 2.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has a DSC thermogram substantially similar to the one set forth in FIG. 3.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has a DSC thermogram with an endotherm having an onset at about 71.5° C.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has a water content from about 0-20% wt. In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form has a water content from about 0-10% wt.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is characterized as having properties: (a) an X-ray powder diffraction (XRPD) pattern substantially the same as shown in FIG. 1; (b) an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta; (c) a thermo-gravimetric analysis (TGA) substantially similar to the one set forth in FIG. 2; (d) a DSC thermogram substantially similar to the one set forth in FIG. 3; (e) a DSC thermogram with an endotherm having an onset at about 71.5° C.; and (f) a water content from about 0-20% wt.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is obtained from methyl isobutyl ketone, ethyl acetate, isopropyl acetate, n-butyl acetate, 2-hexanone, n-butyl methyl ether, t-butyl methyl ether, toluene, acetonitrile/water, tetrahydrofuran (THF)/n-heptane, chloroform/n-heptane, water, or combinations thereof.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is a hydrate. In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is a monohydrate. In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is a channel hydrate.

In some embodiments is a crystalline form of (E)-2-methoxy-4-((8-methylnon-6-enamido)methyl)phenyl 2-((methylamino)methyl)piperidine-1-carboxylate hydrochloride, wherein the crystalline form is anhydrous.

In another aspect, described herein is a pharmaceutical composition comprising a crystalline form of (E)-2-methoxy-4-((8-methylnon-6-enamido)methyl)phenyl 2-((methylamino)methyl)piperidine-1-carboxylate hydrochloride and at least one inactive ingredient selected from pharmaceutically acceptable carriers, diluents, and excipients. In some embodiments, the (E)-2-methoxy-4-((8-methylnon-6-enamido)methyl)phenyl 2-((methylamino)methyl)piperidine-1-carboxylate hydrochloride pharmaceutical composition is formulated for administration to a mammal by intravenous, subcutaneous or other parenteral administration; oral administration, inhalation, nasal administration, topical administration, or ophthalmic administration. In some embodiments, the (E)-2-methoxy-4-((8-methylnon-6-enamido)methyl)phenyl 2-((methylamino)methyl)piperidine-1-carboxylate hydrochloride pharmaceutical composition is in the form of a powder, tablet, a pill, a capsule, a liquid, a suspension, a gel, a dispersion, a solution, an emulsion, an ointment, or a lotion.

In another aspect, described herein is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is post-surgical pain, post amputation pain, chronic post-surgical pain, and traumatic injury pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is post-surgical pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is post-surgical pain from a laparotomy, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is traumatic injury pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is traumatic injury pain from a long bone, short bone, flat bone, or irregular bone fracture. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is traumatic injury pain from a hip or rib fracture. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is chronic post-surgical pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is chronic post-surgical pain after mastectomy or lumpectomy. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is chronic post-surgical pain after thoracotomy. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is chronic post-surgical pain after amputation. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the pain is chronic pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the chronic pain is chronic pain associated with osteoarthritis. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the chronic pain is chronic pain associated with osteoarthritis of the knee. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the chronic pain is chronic musculoskeletal pain. In some embodiments is a method for treating or preventing pain in an individual in need thereof, comprising administering to the individual a therapeutically effective amount of a crystalline form of (E)-2-methoxy-4-((8-methylnon-6-enamido)methyl)phenyl 2-((methylamino)methyl)piperidine-1-carboxylate hydrochloride described herein, wherein the chronic pain is chronic musculoskeletal pain of the lower back. In some embodiments of the methods described herein, further comprises the administration of a second therapeutic agent.

In some embodiments of the methods described herein, the individual is a human.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1. Illustrates an XRPD spectrum of crystalline (E)-2-methoxy-4-((8-methylnon-6-enamido)methyl)phenyl 2-((methylamino)methyl)piperidine-1-carboxylate hydrochloride (Compound 1).

FIG. 2. Illustrates a TGA thermogram of crystalline (E)-2-methoxy-4-((8-methylnon-6-enamido)methyl)phenyl 2-((methylamino)methyl)piperidine-1-carboxylate hydrochloride (Compound 1).

FIG. 3. Illustrates a DSC thermogram of crystalline (E)-2-methoxy-4-((8-methylnon-6-enamido)methyl)phenyl 2-((methylamino)methyl)piperidine-1-carboxylate hydrochloride (Compound 1).

FIG. 4. Illustrates the dynamic vapor sorption (DVS) plot of crystalline (E)-2-methoxy-4-((8-methylnon-6-enamido)methyl)phenyl 2-((methylamino)methyl)piperidine-1-carboxylate hydrochloride (Compound 1) at 25° C. (0% RH-95% RH-0% RH).

FIG. 5. Illustrates X-ray powder diffraction (XRPD) patterns of crystalline (E)-2-methoxy-4-((8-methylnon-6-enamido)methyl)phenyl 2-((methylamino)methyl)piperidine-1-carboxylate hydrochloride (Compound 1), before and after DVS analysis at 25° C. (0% RH-95% RH-0% RH) (top pattern=after DVS, bottom pattern=before DVS).

### DETAILED DESCRIPTION OF THE INVENTION

Described herein are crystalline forms of (E)-2-methoxy-4-((8-methylnon-6-enamido)methyl)phenyl 2-((methylamino)methyl)piperidine-1-carboxylate hydrochloride (Compound 1). Also described herein are pharmaceutical compositions that include the crystalline forms of (E)-2-methoxy-4-((8-methylnon-6-enamido)methyl)phenyl 2-((methylamino)methyl)piperidine-1-carboxylate hydrochloride (Compound 1) and methods of using the compound in the treatment of diseases or conditions.

As described herein, Compound 1 is (E)-2-methoxy-4-((8-methylnon-6-enamido)methyl)phenyl 2-((methylamino)methyl)piperidine-1-carboxylate hydrochloride. “Compound 1” or “(E)-2-methoxy-4-((8-methylnon-6-enamido)methyl)phenyl 2-((methylamino)methyl)piperidine-1-carboxylate hydrochloride” refers to the compound with the following structure:

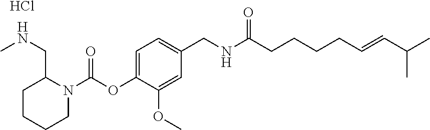 [SMILES: Cl ; CNCC1CCCCN1C(=O)Oc1ccc(CNC(=O)CCCC/C=C/C(C)C)cc1OC]

Pain management in patients after surgery remains insufficient (Pogatzki-Zahn et al., 2012), and there is no ideal way to provide continuous, effective pain relief beyond 12-18 hours after surgery. Systemic pharmacological therapies remain the mainstay of postoperative pain relief, with opioids a key component, especially for moderate-to-severe pain. Systemic opioids are effective, but increase cost and morbidity, especially due to known safety issues such as respiratory depression, gastrointestinal dysfunction, and abuse. Non-opioid analgesics including acetaminophen, nonselective NSAIDs, and selective COX-2 inhibitors are useful for the treatment of light-to-moderate pain and are part of a balanced multimodal pain treatment (Pogatzki-Zahn et al., 2012). These products also have known safety risks.

Site-specific local anesthetic infiltration techniques in which local anesthetic is injected into the tissues around the surgical site are attractive as a component of multi-modal analgesia due to the potential for prevention of post-operative pain, with lower potential safety risks due to the local nature of administration. Treating pain at its source with local anesthetic is highly effective, but limited due to its typically short duration of action. Use of long-acting local anesthetics such as bupivacaine at the surgical site is recommended in the clinical practice guideline on the basis of evidence showing benefit for the surgical procedure in question (Chou et al., 2016). The use of subcutaneous and/or periarticular infiltration of long-acting local anesthetics at the surgical site has been shown to be effective as a component of multimodal analgesia in several surgical procedures, including total knee replacement, arthroscopic knee surgeries, cesarean section, laparotomy, and hemorrhoid surgery, although some studies showed no benefit (Chou et al., 2016).

The utility of conventional local anesthetics is limited by their relatively short duration of action (6-8 hours) and there is a clear need for longer lasting site-specific product whose duration of effect better matches the duration of pain after surgery. Exparel®, an extended release liposomal formulation of bupivacaine, is approved for single-dose infiltration into the surgical site to produce postsurgical analgesia. The analgesic benefit of Exparel® when compared to placebo, however, is limited to 12-24 hours. Moreover, there is limited data to support any benefit of Exparel® over standard bupivacaine.

An additional shortcoming of traditional local anesthetics is their nonselective effect on sensory and motor nerves. The blocking of pain conduction with conventional local anesthetics is accompanied by numbness and motor weakness. The extension of muscle weakness and numbness into the postoperative period would interfere with mobilization and rehabilitation. Another potential drawback is the risk of injury in the absence of sensation. Pain serves as protective reflex and an extended nonselective block of sensory function could result in injury to a numb region of the body Capsaicin, the main ingredient responsible for the hot pungent taste of chili peppers, is an alkaloid found in the Capsicum family. Capsaicin (8-methyl-N-vanillyl-6-nonenamide) is a highly selective agonist for transient receptor potential vanilloid 1 receptor (TRPV1; formerly known as vanilloid receptor 1 (VR1)), a ligand-gated, non-selective cation channel. TRPV1 is preferentially expressed on small-diameter sensory neurons, predominately on C-fibers and to a lesser extent A-delta fibers which specialize in the detection of painful or noxious sensations. TRPV1 responds to stimuli including capsaicin, heat, and extracellular acidification, and will integrate simultaneous exposures to these stimuli. (Caterina M J, Julius D. The vanilloid receptor: a molecular gateway to the pain pathway. Annu Rev Neurosci. 2001. 24:487-517).

TRPV1 agonists, such as capsaicin, have been shown to diminish pain in various settings, but there are problems associated with their use. The initial effects of the activation of TRPV1-expressing (capsaicin-sensitive) nociceptors include burning sensations, hyperalgesia, allodynia, and erythema. However, after prolonged exposure to low-concentration capsaicin or single exposures to high-concentration capsaicin or other TRPV1 agonists, the small-diameter sensory axons become less sensitive to a variety of stimuli, including capsaicin or thermal stimuli. Following the initial activation of nociceptors, capsaicin and other TRPV1 agonists induce a long-lasting, selective reduction in pain responses lasting days to weeks. These later-stage effects of capsaicin are frequently referred to as “desensitization” and are the rationale for the development of capsaicin formulations for the treatment of various pain syndromes and other conditions (Bley, K. R. Recent developments in transient receptor potential vanilloid receptor 1 agonist-based therapies. Expert Opin Investig Drugs. 2004. 13(11): 1445-1456).

In addition, capsaicin and other TRPV1 agonists have very limited water solubility, are extremely potent irritants requiring special equipment when handling and, due to their limited water solubility, are not readily mixed with common drugs that are procured as aqueous solutions.

Therefore, it would be desirable to provide a capsaicin prodrug with: 1) increased water solubility, 2) the potential for reduced or delayed pungency associated with the administration of capsaicin and 3) have the ability to be delivered in a rapid manner (half-life of delivery of capsaicin in less than 30 min) or in a delayed manner (half-life of delivery of capsaicin in greater than 30 min). In addition, it may be desirable to utilize chemical formulations/additives to delay the initiation of prodrug conversion. Furthermore, it would be desirable to provide a capsaicin prodrug that is soluble in aqueous sterile injectable formulations to the intended site of action. Finally, in some cases it would be desirable to deliver another pharmacologically active compound(s) along with a capsaicin prodrug, especially a local anesthetic agent.

Compound 1 releases capsaicin and cyclic urea Compound 2 (2-methylhexahydroimidazo[1,5-a]pyridin-3(2H)-one) under well-defined rates via a pH driven, intra-molecular cyclization release reaction after Compound 1 has been delivered to the body and/or is exposed to specific physiological conditions:

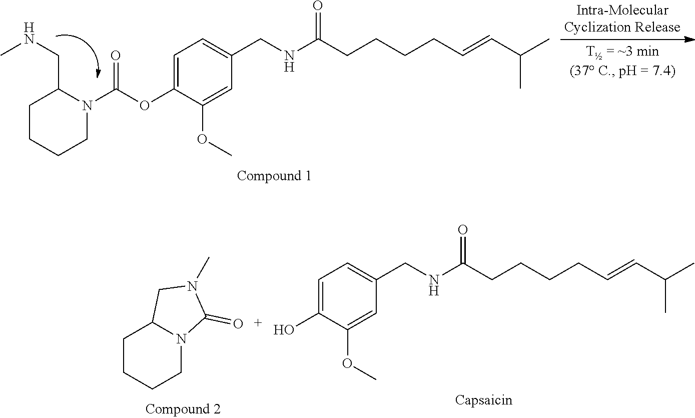

Compound 1 has significantly higher hydrophilicity/water solubility than capsaicin and, hence, is better able to be incorporated into commonly used aqueous formulations. The improved water solubility of Compound 1 is significant when co-delivering other medications, especially when administering multiple sterile agents via injection.

In some embodiments, Compound 1 eliminates the reliance on special requirements for formulations or delivery devices for capsaicin in order to 1) accommodate the very low water solubility of capsaicin and 2) reduce the acute pungency associated with the administration of capsaicin.

In some embodiments, Compound 1 includes the solvent addition forms (solvates). Solvates contain either stoichiometric or non-stoichiometric amounts of a solvent, and are formed during the process of product formation or isolation with pharmaceutically acceptable solvents such as water, ethanol, methanol, tert-butyl methyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In some embodiments, solvates are formed using, but not limited to, Class 3 solvent(s). In some embodiments, solvates are formed using, but not limited to, Class 2 solvent(s). Categories of solvents are defined in, for example, the International Conference on Harmonization of Technical Requirements for Registration of Pharmaceuticals for Human Use (ICH), “Impurities: Guidelines for Residual Solvents Q3C(R6),” (October 2016).

Hydrates are formed when the solvent is water, or alcoholates are formed when the solvent is alcohol. In some embodiments, Compound 1 is hydrated. In some embodiments, Compound 1 is a monohydrate. In some embodiments, Compound 1 is a channel hydrate. In some embodiments, Compound 1 is anhydrous.

In other embodiments, Compound 1 is prepared in various forms, including but not limited to, an amorphous phase, crystalline forms, milled forms, and nano-particulate forms.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein.

### Crystalline Forms

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Crystalline Form 1 of (E)-2-Methoxy-4-((8-methylnon-6-enamido)methyl)phenyl 2-((methylamino)methyl)piperidine-1-carboxylate hydrochloride (Compound 1)

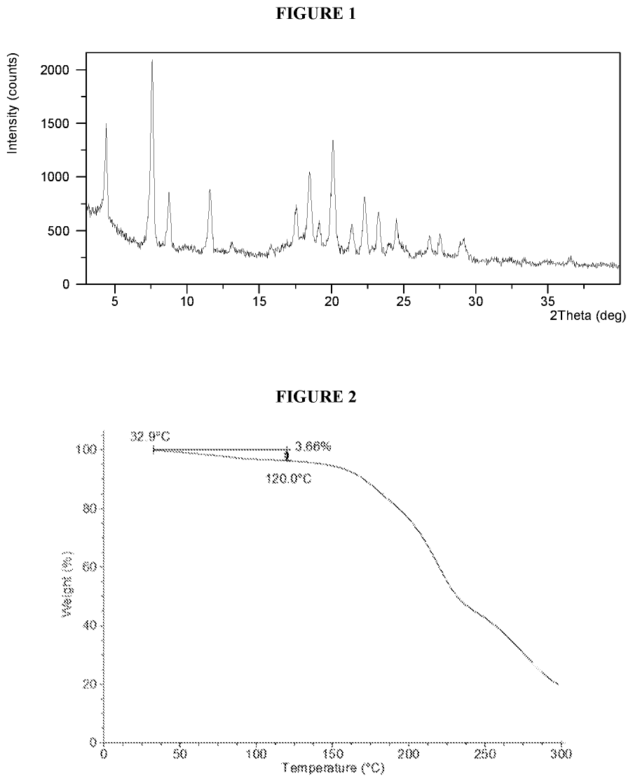

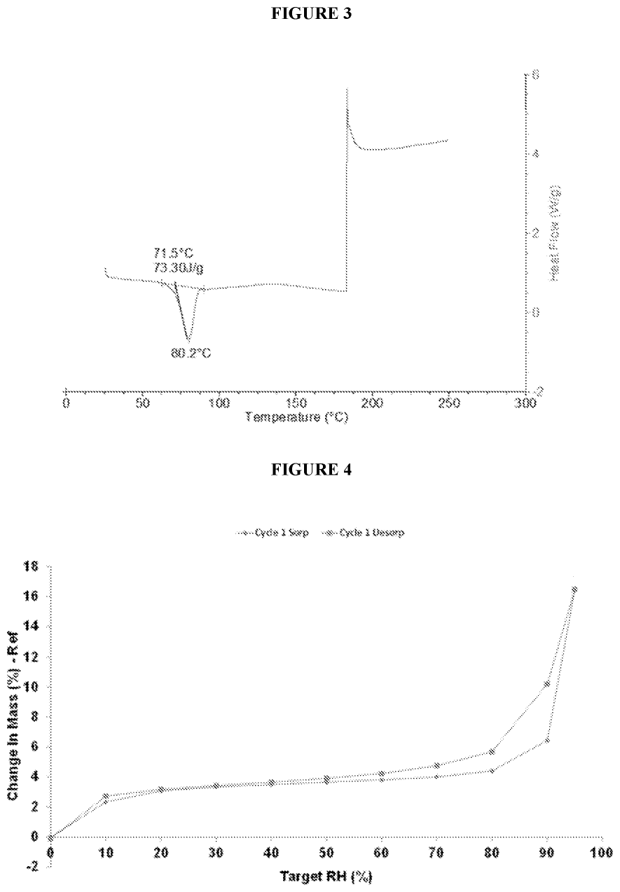

In some embodiments, (E)-2-methoxy-4-((8-methylnon-6-enamido)methyl)phenyl 2-((methylamino)methyl)piperidine-1-carboxylate hydrochloride (Compound 1) is crystalline. In some embodiments, crystalline Compound 1 is characterized as having at least one of the following properties:
(a) an X-ray powder diffraction (XRPD) pattern substantially the same as shown in FIG. 1;
(b) an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with a broad endotherm having an onset at about 71.5° C.;
(f) a water content from about 0-20% wt; or
(g) combinations thereof.

In some embodiments, crystalline Compound 1 is characterized as having at least two of the properties selected from (a) to (f). In some embodiments, crystalline Compound 1 is characterized as having at least three of the properties selected from (a) to (f). In some embodiments, crystalline Compound 1 is characterized as having at least four of the properties selected from (a) to (f). In some embodiments, crystalline Compound 1 is characterized as having at least five of the properties selected from (a) to (f). In some embodiments, crystalline Compound 1 is characterized as having properties (a) to (f).

In some embodiments, crystalline Compound 1 has an X-ray powder diffraction (XRPD) pattern substantially the same as shown in FIG. 1. In some embodiments, crystalline Compound 1 has an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta. In some embodiments, crystalline Compound 1 has a thermo-gravimetric analysis (TGA) thermogram substantially similar to the one set forth in FIG. 2. In some embodiments, crystalline Compound 1 has a DSC thermogram substantially similar to the one set forth in FIG. 3. In some embodiments, crystalline Compound 1, Form 1, has a DSC thermogram with an endotherm having an onset at about 71.5° C.

In some embodiments, crystalline Compound 1, Form 1, has a water content from about 0-20% wt. In some embodiments, crystalline Compound 1, Form 1, has a water content from about 0-16.7% wt. In some embodiments, crystalline Compound 1, Form 1, has a water content from about 0-15% wt. In some embodiments, crystalline Compound 1, Form 1, has a water content from about 0-12.5% wt. In some embodiments, crystalline Compound 1, Form 1, has a water content from 0-10% wt. In some embodiments, crystalline Compound 1, Form 1, has a water content from about 0-7.5% wt. In some embodiments, crystalline Compound 1, Form 1, has a water content from about 0-5% wt. In some embodiments, crystalline Compound 1, Form 1, has a water content from about 0-4% wt. In some embodiments, crystalline Compound 1, Form 1, has a water content from about 0-3.5% wt. In some embodiments, crystalline Compound 1, Form 1, has a water content from about 0-3% wt. In some embodiments, crystalline Compound 1, Form 1, has a water content from about 0-2.5% wt. In some embodiments, crystalline Compound 1, Form 1, has a water content from about 0-2% wt. In some embodiments, crystalline Compound 1, Form 1, has a water content from about 0-1.5% wt. In some embodiments, crystalline Compound 1, Form 1, has a water content from about 0-1% wt. In some embodiments, crystalline Compound 1, Form 1, has a water content from about 0-0.5% wt.

In some embodiments, crystalline Compound 1, Form 1, is obtained from methyl isobutyl ketone, ethyl acetate, isopropyl acetate, n-butyl acetate, 2-hexanone, n-butyl methyl ether, t-butyl methyl ether, toluene, acetonitrile, tetrahydrofuran (THF), n-heptane, chloroform, water, or combinations thereof. In some embodiments, crystalline Compound 1, Form 1, is obtained from a combination of ethyl acetate, acetonitrile, and water. In some embodiments, crystalline Compound 1, Form 1, is obtained from methyl isobutyl ketone and water. In some embodiments, crystalline Compound 1, Form 1, is solvated. In some embodiments, crystalline Compound 1, Form 1, is a hydrate. In some embodiments, crystalline Compound 1, Form 1, is a channel hydrate. In some embodiments, crystalline Compound 1, Form 1, is unsolvated. In some embodiments, crystalline Compound 1, Form 1, is anhydrous.

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. In some embodiments, solvents disclosed herein are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents Q3C(R6),” (October 2016).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cumene, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methyl butyl ketone, methylcyclohexane, methyl isobutyl ketone, N-methylpyrrolidone, nitromethane, pyridine, sulfolane, tetrahydrofuran, tetralin, toluene, 1,1,2-trichloroethene and xylene.

Class 3 solvents, which possess low toxicity, include: acetic acid, acetone, anisole, 1-butanol, 2-butanol, butyl acetate, tert-butyl methyl ether (MTBE), dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, 2-methyl-1-propanol, pentane, 1-pentanol, 1-propanol, 2-propanol, propyl acetate, and triethylamine.

Residual solvents in active pharmaceutical ingredients (APIs) originate from the manufacture of APIs. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound 1 comprise an organic solvent(s). In some embodiments, compositions comprising Compound 1 comprise a residual amount of an organic solvent(s). In some embodiments, compositions comprising Compound 1 comprise a residual amount of a Class 3 solvent. In some embodiments, the organic solvent is a Class 3 solvent. In some embodiments, the Class 3 solvent is selected from the group consisting of acetic acid, acetone, anisole, 1-butanol, 2-butanol, butyl acetate, tert-butyl methyl ether (MTBE), dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, 2-methyl-1-propanol, pentane, 1-pentanol, 1-propanol, 2-propanol, propyl acetate, and triethylamine. In some embodiments, the Class 3 solvent is selected from the group consisting of acetone, ethyl acetate, isopropyl acetate, tert-butyl methyl ether, heptane, isopropanol, and ethanol.

In some embodiments, compositions comprising Compound 1 comprise a residual amount of a Class 2 solvent. In some embodiments, the organic solvent is a Class 2 solvent. In some embodiments, the Class 2 solvent is selected from the group consisting of acetonitrile, chlorobenzene, chloroform, cumene, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methyl butyl ketone, methylcyclohexane, methyl isobutyl ketone, N-methylpyrrolidone, nitromethane, pyridine, sulfolane, tetrahydrofuran, tetralin, toluene, 1,1,2-trichloroethene and xylene. In some embodiments, the Class 2 solvent is selected from the group consisting of acetonitrile, tetrahydrofuran, and toluene. In some embodiments, the Class 2 solvent is acetonitrile.

In some embodiments, compositions comprising Compound 1 comprise a residual amount of a solvent for which no adequate toxicological data were found. In some embodiments, the organic solvent is a solvent for which no adequate toxicological data were found. In some embodiments, the solvent is selected from the group consisting of 2-butanone and 2-methyltetrahydrofuran.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features. The term “about” when referring to a number or a numerical range means that the number or numerical range referred to is an approximation within experimental variability (or within statistical experimental error), and thus the number or numerical range may vary between 1% and 15% of the stated number or numerical range.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Bioavailability” refers to the percentage of Compound 1 dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which Compound 1 is absorbed into the general circulation when the pharmaceutical composition is taken orally as compared to intravenous injection.

“Blood plasma concentration” refers to the concentration of Compound 1 in the plasma component of blood of a subject. It is understood that the plasma concentration of Compound 1 may vary significantly between subjects, due to variability with respect to metabolism and/or possible interactions with other therapeutic agents. In accordance with one embodiment disclosed herein, the blood plasma concentration of Compound 1 may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of Compound 1 may vary from subject to subject.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Compound 1, age, weight, general condition of the subject, the condition being treated, the severity of the condition being treated, and the judgment of the prescribing physician. By way of example only, therapeutically effective amounts may be determined by a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder, or condition. An “enhancing-effective amount,” as used herein, refers to an amount adequate to enhance the effect of a therapeutic agent in the treatment of a disease, disorder, or condition. When used in a patient, amounts effective for this use will depend on the severity and course of the disease, disorder, or condition, previous therapy, the patient's health status and response to the drugs, and the judgment of the treating physician.

The term “subject” or “individual” as used herein, refers to an animal which is the object of treatment, observation or experiment. By way of example only, a subject may be, but is not limited to, a mammal including, but not limited to, a human.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

### Pharmaceutical Compositions/Formulations

In some embodiments, Compound 1 described herein is formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, Compound 1 described herein is administered either alone or in combination with pharmaceutically acceptable carriers, excipients or diluents, in a pharmaceutical composition. Administration of Compound 1 described herein, and pharmaceutical compositions thereof, can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, Compound 1 can be administered locally to the area in need of treatment, by for example, local infusion during surgery, topical application such as creams or ointments, injection, catheter, or implant. The administration can also be by direct injection (e.g., infiltration or instillation) at the site of a diseased tissue or organ.

In some embodiments, Compound 1 pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

### Methods

In some embodiments, described herein is a method for treating or preventing pain in an individual in need thereof, comprising administering to the individual a therapeutically effective amount of a crystalline form of Compound 1 described herein. In some embodiments, described herein is a method for treating pain in an individual in need thereof, comprising administering to the individual a therapeutically effective amount of a crystalline form of Compound 1 described herein. Some embodiments, described herein is a method for preventing pain in an individual in need thereof, comprising administering to the individual a therapeutically effective amount of a crystalline form of Compound 1 described herein. In some embodiments is a method for treating or preventing pain in an individual in need thereof, comprising administering to the individual a therapeutically effective amount of a crystalline form of Compound 1 described herein, wherein the pain is post-surgical pain, post amputation pain, chronic post-surgical pain, and traumatic injury pain. In some embodiments is a method for treating or preventing pain in an individual in need thereof, comprising administering to the individual a therapeutically effective amount of a crystalline form of Compound 1 described herein, wherein the pain is post-surgical pain. In some embodiments is a method for treating or preventing pain in an individual in need thereof, comprising administering to the individual a therapeutically effective amount of a crystalline form of Compound 1 described herein, wherein the pain is post-surgical pain from a laparotomy, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures. In some embodiments is a method for treating or preventing pain in an individual in need thereof, comprising administering to the individual a therapeutically effective amount of a crystalline form of Compound 1 described herein, wherein the pain is traumatic injury pain. In some embodiments is a method for treating or preventing pain in an individual in need thereof, comprising administering to the individual a therapeutically effective amount of a crystalline form of Compound 1 described herein, wherein the pain is traumatic injury pain from a long bone, short bone, flat bone, or irregular bone fracture. In some embodiments is a method for treating or preventing pain in an individual in need thereof, comprising administering to the individual a therapeutically effective amount of a crystalline form of Compound 1 described herein, wherein the pain is traumatic injury pain from a hip or rib fracture. In some embodiments is a method for treating or preventing pain in an individual in need thereof, comprising administering to the individual a therapeutically effective amount of a crystalline form of Compound 1 described herein, wherein the pain is chronic post-surgical pain. In some embodiments is a method for treating or preventing pain in an individual in need thereof, comprising administering to the individual a therapeutically effective amount of a crystalline form of Compound 1 described herein, wherein the pain is chronic post-surgical pain after mastectomy or lumpectomy. In some embodiments is a method for treating or preventing pain in an individual in need thereof, comprising administering to the individual a therapeutically effective amount of a crystalline form of Compound 1 described herein, wherein the pain is chronic post-surgical pain after thoracotomy. In some embodiments is a method for treating or preventing pain in an individual in need thereof, comprising administering to the individual a therapeutically effective amount of a crystalline form of Compound 1 described herein, wherein the pain is chronic post-surgical pain after amputation. In some embodiments is a method for treating or preventing pain in an individual in need thereof, comprising administering to the individual a therapeutically effective amount of a crystalline form of Compound 1 described herein, wherein the pain is chronic pain. In some embodiments is a method for treating or preventing pain in an individual in need thereof, comprising administering to the individual a therapeutically effective amount of a crystalline form of Compound 1 described herein, wherein the chronic pain is chronic pain associated with osteoarthritis. In some embodiments is a method for treating or preventing pain in an individual in need thereof, comprising administering to the individual a therapeutically effective amount of a crystalline form of Compound 1 described herein, wherein the chronic pain is chronic pain associated with osteoarthritis of the knee. In some embodiments is a method for treating or preventing pain in an individual in need thereof, comprising administering to the individual a therapeutically effective amount of a crystalline form of Compound 1 described herein, wherein the chronic pain is chronic musculoskeletal pain. In some embodiments is a method for treating or preventing pain in an individual in need thereof, comprising administering to the individual a therapeutically effective amount of a crystalline form of Compound 1 described herein, wherein the chronic pain is chronic musculoskeletal pain of the lower back. In some embodiments of the methods described herein, further comprises the administration of a second therapeutic agent.

### Methods of Dosing and Treatment Regimens

The compositions described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder, or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered, if necessary. Subsequently, the dosage or the frequency of administration, or both, can be reduced, as a function of the symptoms, to a level at which the improved disease, disorder, or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02 mg to about 5000 mg per day, in some embodiments, about 1 mg to about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### EXAMPLES

### List of Abbreviations

As used throughout the description of the invention, the following abbreviations, unless otherwise indicated, shall be understood to have the following meanings:
ACN or MeCN acetonitrile
Bn benzyl
BOC or Boc tert-butyl carbamate
t-Bu tert-butyl
Cy cyclohexyl
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
eq or equiv equivalent(s)
Et ethyl
Et₂O diethyl ether
EtOH ethanol
EtOAc ethyl acetate
GC gas chromatography
h hour(s)
HPLC high performance liquid chromatography
IPA isopropyl alcohol
IPAc isopropyl acetate
KF Karl Fischer
Me methyl
MeOH methanol
MS mass spectroscopy
MEK methyl ethyl ketone
MIBK methyl isobutyl ketone
MTBE methyl t-butyl ether
min minutes
MsOH methanesulfonic acid
NMR nuclear magnetic resonance
RP-HPLC reverse phase-high performance liquid chromatography
RT room temperature
TFA trifluoroacetic acid
THF tetrahydrofuran
TLC thin layer chromatography

### Example 1: Preparation of Amorphous (E)-2-Methoxy-4-((8-methylnon-6-enamido)methyl)phenyl 2-((methylamino)methyl)piperidine-1-carboxylate hydrochloride (Compound 1)

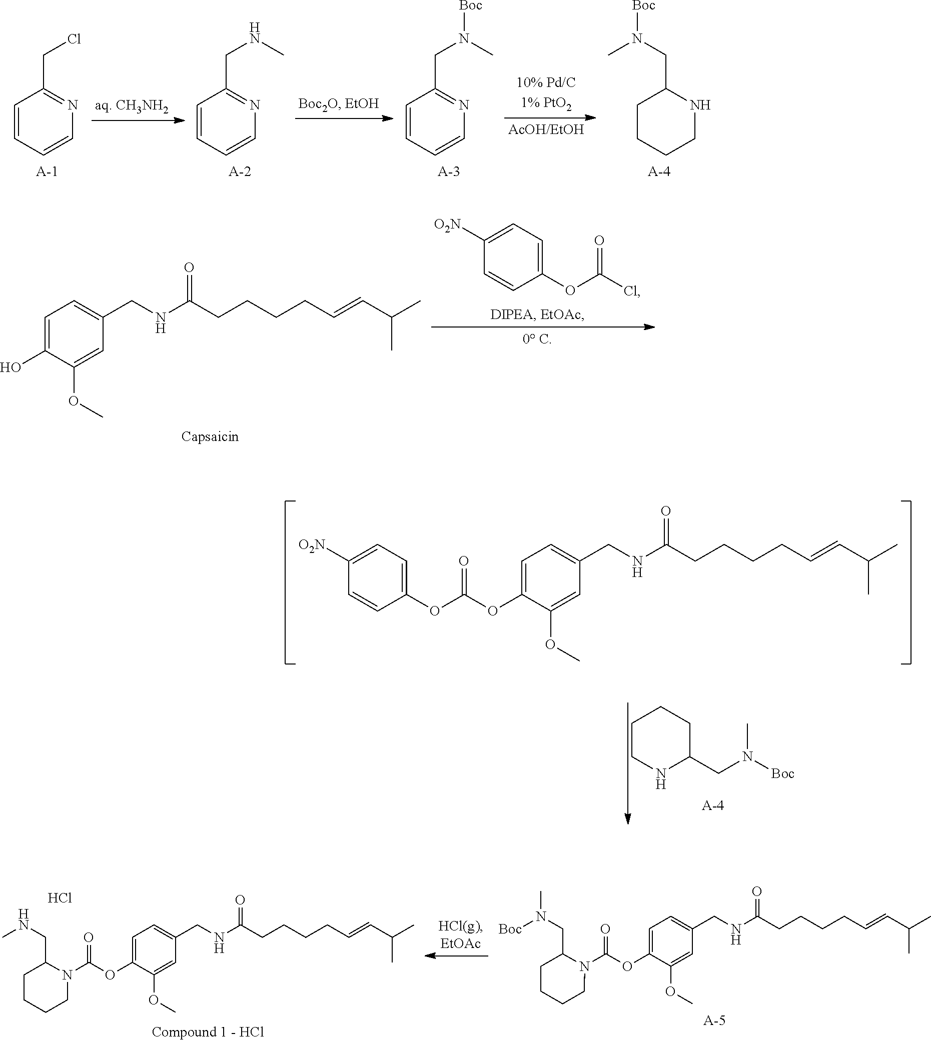

2-(Chloromethyl)pyridine (1.0 eq) was dissolved in water and added dropwise to a solution of 40% aq. Methylamine (20.0 eq) at <5° C. over 2.5 h, maintaining the reaction temperature at 5° C. After the addition was complete, the reaction was warmed to room temperature over 30 min, then concentrated to red oil/solid. This was dissolved in water and cooled to 10° C. Then cold 50% aq. NaOH (4° C., 2.5 eq) was added over 20 min, and the suspension warmed to 40° C. The biphasic suspension was then cooled to room temperature and the salt was filtered. The filtrate was removed and the layers were separated. The filter cake was rinsed with iPrOAc and the iPrOAc filtrate was used to extract the aqueous portion of the initial filtrate. The iPrOAc layer was concentrated with a rotary evaporator and the resulting oil was combined with the organic portion of the initial filtrate. The resulting red oil was concentrated under high vacuum overnight. The flask was then fitted with a distillation head and the product distilled with a B.P. of 67° C. @ 5 Torr to afford compound A-2.

Compound A-2 (1.0 eq) was dissolved in EtOH and cooled to 10° C. Then a solution of Boc₂O (1.0 eq) in EtOH was added drop-wise over 60 min, maintaining the reaction temperature <20° C. Gas was evolved during the addition. After the addition, the solution was warmed to room temperature and stirred for 60 minutes, until gas evolution ceased. Then HPLC indicated complete conversion to A-3. The crude product was used in the following reaction without further manipulation.

To the crude product mixture from the synthesis of A-3 (see above) was added acetic acid (10 eq.), followed by catalyst, 10 wt (wet) Pd/C (10 wt %/C) and 1 wt % PtO₂ (10 wt %/C). The suspension was placed under a H₂ atmosphere and shaken under 55 PSI H₂ with a Parr shaker for 9 days. The suspension was filtered through celite under argon and concentrated with a rotary evaporator. The mixture was further concentrated with high vacuum overnight to afford A-4 as the acetate salt.

To a reaction flask charged with capsaicin (1 eq) and ethyl acetate, the solution was cooled to 0-10° C. and DIPEA (3 eq) was added followed by the addition of nitrophenylchloroformate (1.0 eq) as a solution in ethyl acetate at 0-10° C. The resulting mixture was stirred at 0-10° C. for 15 min. Next, HOBt (0.1 eq) was added, followed by A-4 free base (1.2 eq) at 0-10° C. The resulting mixture was stirred overnight after warming to room temperature. The reaction mixture was worked up by successive extractions with 1 M aq. NaOH (3x), 1 M aq. HCl, water and finally brine solution. The resulting organic layer was removed, dried over sodium sulfate and filtered to afford A-5 as the ethyl acetate solution. The crude product was used in the following reaction without further manipulation.

To the crude product mixture from the synthesis of A-5 (see above), the mixture was cooled to 0-10° C. with stirring and sparged with HCl (g) for approximately 30 seconds. The resulting mixture was stirred at 0-10° C. for approximately 2 h. The resulting mixture was concentrated to afford amorphous Compound 1.

### Example 1a: Preparation of Crystalline Compound 1, Form 1 (methyl iso-butyl ketone)

Amorphous Compound 1 (16.15 g) was charged to the reactor and suspended in MIBK (18 vol, 290 mL). The suspension was heated to 54° C. and a clear solution was obtained. The mixture was cooled to below 40° C. and seed crystals were added (150 mg, 1 wt %). The suspension was cooled to 23° C. resulting in a thick slurry. The mixture is filtered and dried to afford crystalline Compound 1, Form 1.

### Example 1b: Preparation of Crystalline Compound 1, Form 1 (Acetonitrile, Water, Ethyl Acetate)

Amorphous Compound 1 (100 g) was charged to the reactor and dissolved in ACN(230-240 mL) and water (30 mL) is added. The mixture is adjusted to 25-30° C. and EtOAc (1.4 L) is added. To the mixture is added Compound 1 seed crystals were added (100 mg) and is stirred for 2 hrs. Additional EtOAc (710-720 mL) is added and the resulting slurry is stirred. The mixture is filtered, washed and dried to afford crystalline Compound 1, Form 1.

### Example 2: Preparation of Crystalline Compound 1, Form 1 (by Slow Evaporation)

Amorphous Compound 1 (10 mg) was dissolved in EtOAc (0.5-2.0 mL) in a 3-mL glass vial. The visually clear solution was subjected to evaporation at RT with the vial covered by Parafilm® (3 holes). After evaporation for ˜3 days, obtained solid was isolated to afford crystalline Compound 1, Form 1.

### Example 3A: Preparation of Crystalline Compound 1, Form 1 (by Slurry at 5° C.)

Amorphous Compound 1 (12 mg) was suspended in EtOAc or THF/n-heptane (1:2, v/v) (0.3-0.6 mL) in a 1.5-mL glass vial at 5° C. After the suspension was stirred (1000 rpm) magnetically for about 2 days, the solid was isolated to afford crystalline Compound 1, Form 1.

### Example 3B: Preparation of Crystalline Compound 1, Form 1 (by Slurry at 5° C.)

Amorphous Compound 1 (5 mg) was suspended in 0.35 mL of isopropyl acetate, n-butyl acetate, 2-hexanone, or n-butyl methyl ether in a 1.5-mL glass vial. After the suspension was stirred (1000 rpm) magnetically for about 4 days, the remaining solids were isolated to afford crystalline Compound 1, Form 1.

### Example 4: Preparation of Crystalline Compound 1, Form 1 (by Anti-solvent Addition)

Amorphous Compound 1 (8 mg) was dissolved in chloroform (0.25 mL) to obtain a clear solution, and the solution was magnetically stirred followed by addition of n-heptane (0.1 mL) in increments until precipitation was observed or the volume of anti-solvent reached 2 mL. The obtained precipitate was isolated afford crystalline Compound 1, Form 1.

### Example 5: Preparation of Crystalline Compound 1, Form 1 (by Slurry with Temperature Cycling at 5-50° C.)

Crystalline Compound 1, Form 1, (5 mg) was suspended in 0.35 mL solvent (acetone, THF, 1,4-dioxane, MTBE, or toluene) and then stirred with temperature cycling (50-5-50-5° C., 0.1° C./min). The final temperature was kept at 5° C. Residual solids were isolated to check by XRPD. Crystalline Form 1 was detected from MTBE and toluene systems, and it was also obtained after addition of n-heptane in acetone, THF and 1,4-dioxane systems.

### Example 6: X-ray Powder Diffraction (XRPD)

X-ray powder diffraction studies were performed using an X-ray powder diffractometer with the following instrument parameters:

| Parameter | Value | |
|---|---|---|
| Detector | PSD: Lynx Eye | |
| Scan Type | Lock Coupled | |
| Scan Mode | Continuous | |
| Generator voltage | 40 | kV |
| Generator Current | 40 | mA |
| Scan range (2 theta) | 2.75°-40° | |
| Divergence Slit | V12.000 | mm |
| Time per step | ~0.4 | sec |
| Step Increment | ~0.009° | |
| Number of Steps | 4053 | |
| Lynx-Iris Position | 1.00 | mm |

| | |
|---|---|
| Synchronous Rotation | on |

XRPD analysis of Compound 1, Form 1, (FIG. 1) showed Compound 1 to be crystalline with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta.

### Example 7: Thermogravimetric Analysis (TGA)

TGA data were collected using a Q500/5000 TGA from TA Instruments with the following instrument parameters:

| Method | Ramp |
|---|---|
| Sample pan | Platinum, open |
| Temperature | RT—desired temperature |
| Heating rate | 10° C./min |
| Purge gas | N₂ |

TGA of crystalline Compound 1, Form 1, (FIG. 2) showed a weight loss of 3.7% up to 120° C.

### Example 8: Differential Scanning Calorimetry (DSC)

DSC was performed using a Q20 DSC from TA Instruments with the following instrument parameters:

| Method | Ramp |
|---|---|
| Sample pan | Aluminum, hermetic |
| Temperature | 35° C.-160° C. |
| Heating rate | 10° C./min |
| Purge gas | N₂ |

DSC result of crystalline Compound 1, Form 1, (FIG. 2) showed a broad endothermic peak with an onset at about 73.6° C.

### Example 9: Dynamic Vapor Sorption (DVS)

DVS test was performed using an SMS (Surface Measurement Systems) DVS Intrinsic instrument. The relative humidity at 25° C. were calibrated against deliquescence point of LiCl, Mg(NO₃)₂ and KCl. The following instrument parameters were used:

| Temperature | 25° C. | |
|---|---|---|
| Sample size | 10~20 | mg |
| Gas and flow rate | N₂, 200 mL/min | |
| dm/dt | 0.002%/min | |
| Min. dm/dt stability | 10 | min |
| duration | | |
| Max. equilibrium time | 180 | min |

| | |
|---|---|
| RH range | 0% RH-95% RH |
| RH step size | 10% RH from 0% RH to |
| | 90% RH and 90% RH to |
| | 0% RH 5% RH from |
| | 90% RH to 95% RH and |
| | 95% RH to 90% RH |

DVS test was performed on crystalline Compound 1, Form 1, to investigate its form stability as a function of relative humidity. The DVS test was first conducted at 25° C. with humidity cycled between 0% RH-95% RH-0% RH.

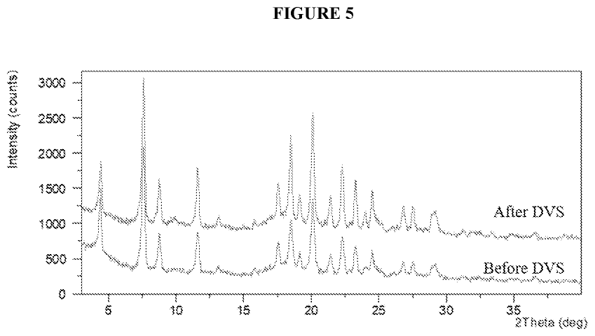

As shown in FIG. 4, the moisture content reached 16.5% wt at 95% RH. The crystal forms after DVS test were detected by XRPD. A relative stable platform at ˜3.5% wt between 20% RH and 70% RH, indicated crystalline Compound 1, Form 1, is a potential hydrate (the theoretical water content in HCl salt monohydrate is 3.5% wt). As shown in FIG. 5, no form change occurred for crystalline Compound 1, Form 1, before and after the DVS test.

### Example 10: Ion Chromatography (IC)

IC test was used to test for chloride content. The following parameters were used:

| Column | IonPac AS18 Analytical Column | |
|---|---|---|
| | (4 × 250 mm) | |
| Mobile phase | 25 | mM NaOH |
| Injection volume | 25 | μL |
| Flow rate | 1.0 | mL/min |
| Cell temperature | 35° C. | |
| Column temperature | 35° C. | |
| Current | 80 | mA |
| Run time | 18.0 | mins for Cl— |

IC data showed the chloride content of crystalline Compound 1, Form 1, was 7.0% wt, which was close to the content in the amorphous starting material (7.4% wt) and matched well with the theoretical value of mono-HCl salt at 7.2% wt.

We claim:

1. A crystalline form of (E)-2-methoxy-4-((8-methylnon-6-enamido)methyl)phenyl 2-((methylamino)methyl)piperidine-1-carboxylate hydrochloride, or solvate thereof.

2. The crystalline form of claim 1, wherein the crystalline form of (E)-2-methoxy-4-((8-methylnon-6-enamido)methyl)phenyl 2-((methylamino)methyl)piperidine-1-carboxylate hydrochloride is Form 1 having at least one of the following properties:
(a) an X-ray powder diffraction (XRPD) pattern substantially the same as shown in FIG. 1;
(b) an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with a broad endotherm having an onset at about 71.5° C.;
(f) a water content from about 0-20% wt; or
(g) combinations thereof.

3. The crystalline form of claim 1 or claim 2, wherein the crystalline form has an X-ray powder diffraction (XRPD) pattern substantially the same as shown in FIG. 1.

4. The crystalline form of claim 1 or claim 2, wherein the crystalline form has an X-ray powder diffraction (XRPD) pattern with characteristic peaks at about 4.4° 2-Theta, 7.6° 2-Theta, 8.8° 2-Theta, 11.6° 2-Theta, 18.5° 2-Theta, 20.1° 2-Theta, and 22.3° 2-Theta.

5. The crystalline form of claim 1 or claim 2, wherein the crystalline form has a thermo-gravimetric analysis (TGA) substantially similar to the one set forth in FIG. 2.

6. The crystalline form of claim 1 or claim 2, wherein the crystalline form has a DSC thermogram substantially similar to the one set forth in FIG. 3.

7. The crystalline form of claim 1 or claim 2, wherein the crystalline form has a DSC thermogram with an endotherm having an onset at about 71.5° C.

8. The crystalline form of claim 1 or claim 2, wherein the crystalline form has a water content from about 0-20% wt.

9. The crystalline form of claim 8, wherein the crystalline form has a water content from about 0-10% wt.

10. The crystalline form of claim 2, wherein the crystalline form is characterized as having properties (a), (b), (c), (d), (e), and (f).

11. The crystalline form of any one of claims 1-10, wherein the crystalline form is obtained from methyl isobutyl ketone, ethyl acetate, isopropyl acetate, n-butyl acetate, 2-hexanone, n-butyl methyl ether, t-butyl methyl ether, toluene, acetonitrile/water, tetrahydrofuran (THF)/n-heptane, chloroform/n-heptane, water, or combinations thereof.

12. The crystalline form of any one of claims 1-11, wherein the crystalline form is a hydrate.

13. The crystalline form of any one of claims 1-12, wherein the crystalline form is a monohydrate.

14. The crystalline form of any one of claims 1-13, wherein the crystalline form is a channel hydrate.

15. The crystalline form of any one of claims 1-11, wherein the crystalline form is anhydrous.

16. A pharmaceutical composition comprising the crystalline form of any one of claims 1-15, and at least one inactive ingredient selected from pharmaceutically acceptable carriers, diluents, and excipients.

17. The pharmaceutical composition of claim 16 formulated for oral, intravenous, intramuscular, or subcutaneous administration.

18. A method for treating or preventing pain in an individual in need thereof, comprising administering to the individual a therapeutically effective amount of a crystalline form of any one of claims 1-15.

19. The method of claim 18, wherein the pain is post-surgical pain, post amputation pain, chronic post-surgical pain, and traumatic injury pain.

20. The method of claim 19, wherein the pain is post-surgical pain.

21. The method of claim 20, wherein the post-surgical pain is pain from a laparotomy, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures.

22. The method of claim 19, wherein the pain is traumatic injury pain.

23. The method of claim 22, wherein the traumatic injury pain is pain from a long bone, short bone, flat bone, or irregular bone fracture.

24. The method of claim 22, wherein the traumatic injury pain is pain from a hip or rib fracture.

25. The method of claim 19, wherein the pain is chronic post-surgical pain.

26. The method of claim 25, wherein the pain is chronic post-surgical pain after mastectomy or lumpectomy.

27. The method of claim 25, wherein the pain is chronic post-surgical pain after thoracotomy.

28. The method of claim 25, wherein the pain is chronic post-surgical pain after amputation.

29. The method of claim 18, wherein the pain is chronic pain.

30. The method of claim 29, wherein the chronic pain is chronic pain associated with osteoarthritis.

31. The method of claim 30, wherein the chronic pain is chronic pain associated with osteoarthritis of the knee.

32. The method of claim 29, wherein the chronic pain is chronic musculoskeletal pain.

33. The method of claim 32, wherein the chronic pain is chronic musculoskeletal pain of the lower back.

34. The method of any one of claims 18-33, further comprising the administration of a second therapeutic agent.
